(19) United States
(12) Patent Application Publication
(10) Pub. No.: US 2009/0038974 A1
(43) Pub. Date: Feb. 12, 2009

(54) Far infrared ray cup

(76) Inventor: Hsin-Mao Hsieh, Pingtung City (TW)

Correspondence Address: ELIZABETH CHIEN-HALE, 40087 MISSION BLVD. BOX 367, FREMONT, CA, 94539, US

(21) Appl. No.: 11/890,567

(22) Filed: Aug. 7, 2007

(51) Int. Cl. A61B 18/00

(52) U.S. Cl. 206/438

(57) ABSTRACT

A far infrared cup is mounted with far infrared ray micro particle layers on both the outer surface and the inner surface. A heater is mounted at the bottom of the cup body to stimulate the far infrared ray micro particles to radiate far infrared rays. The liquid in the far infrared ray cup comprises water that interacts with the far infrared rays to become smaller and more easily absorbed by a human body to improve circulation and metabolism and be beneficial for human health.

### BACKGROUND OF THE INVENTION

1. Field of the Invention

The present invention relates to a far infrared cup, especially to a far infrared cup mounted with far infrared ray micro particle layers to benefit human health.

2. Description of the Related Art

Far infrared rays have a wavelength close to wavelength of the human body. Therefore, the far infrared rays tends to be absorbed easily by human cells, for example, skin and bone marrow cells of humans. The far infrared ray is capable of interacting with water molecules of the human body, improving metabolism and relieving the nervous system. The far infrared ray can also eliminate subcutaneous lipids, improve a person's immune system and further comfort and relieve pain. Accordingly, the far infrared ray is an important light for both animals and plants.

In addition, drinking water is one of the essential activities of human. Sufficient amounts of water helps the metabolism of human body. However, the weather of the earth has been changed these days, the water source on the planet is getting shortened and becomes more valuable. In order to make the drinking water healthier and to be absorbed easily by the human cells, to develop a technology of improve the quality of drinking water is essential and important to the market.

### SUMMARY OF THE INVENTION

The main object of the present invention is to provide a far infrared ray cup mounted with far infrared ray micro particle layers on both the inner surface and the outer surface of the far infrared ray cup. A heater is mounted at the bottom of the cup body to stimulate far infrared ray micro particles to radiate far infrared rays. The liquid in the far infrared ray cup comprises water that interacts with the far infrared rays to become smaller and more easily absorbed by a human body to improve metabolism and circulation.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of the infrared ray cup in accordance with the present invention; and

FIG. 2 is a front view in partial section of the infrared ray cup in FIG. 1.

### DETAILED DESCRIPTION OF THE PREFERRED EMBODIMENTS

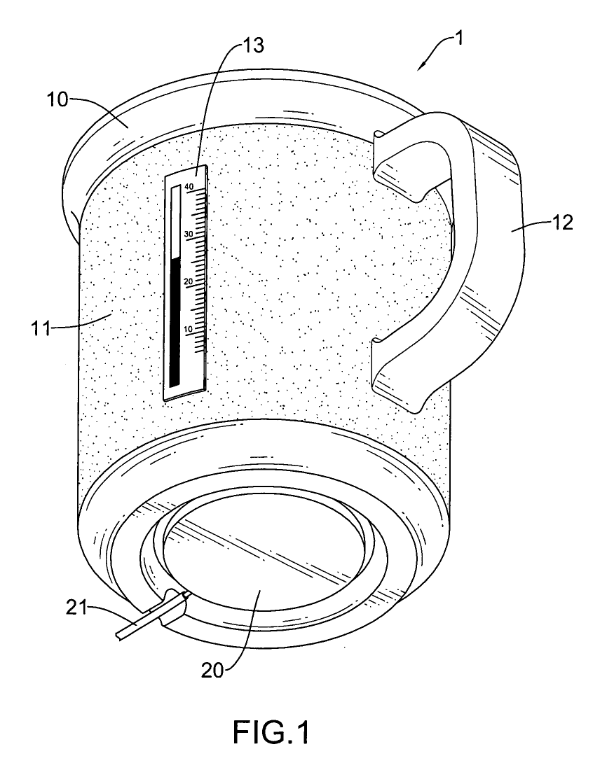

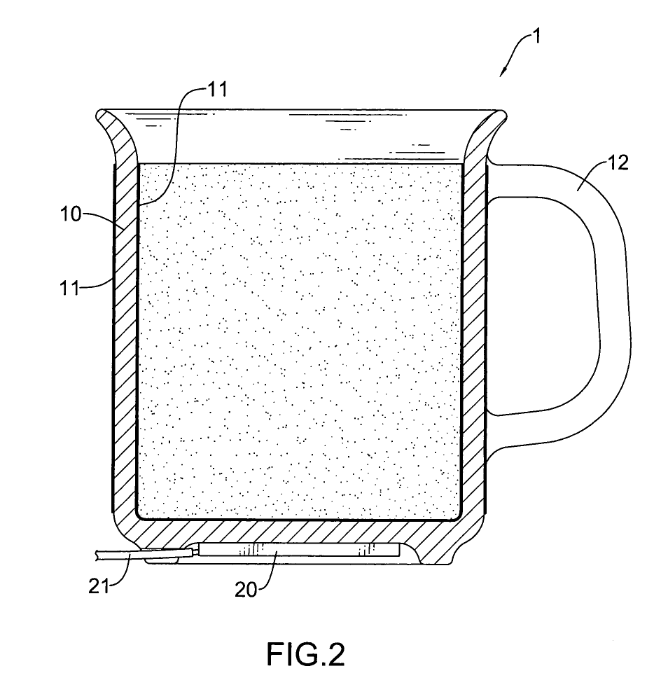

With reference to FIGS. 1 and 2, the infrared ray cup in accordance with the present invention comprises a cup body (10), an infrared ray micro particle layer (11) and a heater (20).

The cup body (10) is an open container comprising a bottom, an outer surface, an inner surface, a handle (12) and an optional thermometer (13). The handle (21) is mounted on the outer surface of the cup body (10). The thermometer (13) is mounted on the outside of the cup body (10).

The far infrared ray micro particle layer (11) is mounted both on the inner surface and the outer surface of the cup body (10) and can be ceramic powder, bamboo charcoal powder, far infrared ray zirconia powder or the like. When the far infrared ray micro particle layer (11) is heated, the particles of the layer radiate far infrared ray energy.

The heater (20) is an electrical heating device and is connected to a power source and may have a cord (21). The cord (21) is mounted on the heater (20) and connects the heater (20) to a power source. The power source may be batteries or main power.

When a liquid comprising water molecules is poured into the cup body (10), the power source is connected to power the heater (20). Heat is conducted through the cup body (10) to the far infrared ray micro particle layer (11) that is stimulated to radiate far infrared rays. The far infrared rays radiated from the far infrared ray micro particle layer (11) interact with the liquid in the cup body (10). The water molecules of the liquid in the cup body (10) is modified and reduces, and is therefore more easily absorbed by human cells to improve circulation and metabolism of a human body.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A far infrared ray cup, comprising:
a cup body being an open container and having
a bottom;
an outer surface;
an inner surface; and
a handle
a far infrared ray micro particle layer mounted on both the outer surface and the inner surface; and
a heater.

2. The far infrared ray cup as claim in claim 1, wherein the cup body further comprises a thermometer mounted on the outer surface of the body.

3. The far infrared ray cup as claim in claim 1, wherein the heater further comprises a cord.

4. The far infrared ray cup as claim in claim 1, wherein the far infrared ray micro particle layer is ceramic powder.

5. The far infrared ray cup as claim in claim 2, wherein the far infrared ray micro particle layer is ceramic powder.

6. The far infrared ray cup as claim in claim 3, wherein the far infrared ray micro particle layer is ceramic powder.

7. The far infrared ray cup as claim in claim 1, wherein the far infrared ray micro particle layer is bamboo charcoal powder.

8. The far infrared ray cup as claim in claim 2, wherein the far infrared ray micro particle layer is bamboo charcoal powder.

9. The far infrared ray cup as claim in claim 3, wherein the far infrared ray micro particle layer is bamboo charcoal powder.

10. The far infrared ray cup as claim in claim 1, wherein the far infrared ray micro particle layer is far infrared ray zirconia powder.

11. The far infrared ray cup as claim in claim 2, wherein the far infrared ray micro particle layer is far infrared ray zirconia powder.

12. The far infrared ray cup as claim in claim 3, wherein the far infrared ray micro particle layer is far infrared ray zirconia powder.
